(19) United States
(12) Patent Application Publication
(10) Pub. No.: US 2011/0125064 A1
(43) Pub. Date: May 26, 2011

(54) SMART CLOTHING FOR MOTION PHYSIOLOGICAL MEASUREMENT AND DYNAMICAL STABLE APPARATUS THEREOF

(75) Inventor: TIEN-WEI SHYR, Taichung (TW)

(73) Assignee: Feng Chia University, Taichung (TW)

(21) Appl. No.: 12/889,034

(22) Filed: Sep. 23, 2010

(30) Foreign Application Priority Data
Nov. 20, 2009 (TW) 098139638

(51) Int. Cl. A61B 5/103

(52) U.S. Cl. 600/595

(57) ABSTRACT

A dynamical stable apparatus of a smart clothing for motion physiological measurement includes at least one physiological sensing module. The physiological sensing module includes a carrier, at least one physiological sensor and a first tension adjusting component. The carrier has a surface for contacting with a body of a user, and the surface is coated with a slide resistant material. The physiological sensor is disposed on the surface of the carrier and configured for measuring a motion physiological signal of the user. The first tension adjusting component is disposed on two opposite ends of the carrier and configured for adjusting a tension of the carrier so as to fix the carrier on the body of the user. When the user wears the smart clothing for motion physiological measurement to measure a motion physiological signal, a stable motion physiological signal can be obtained.

### CROSS-REFERENCE TO RELATED APPLICATION

This application claims the priority benefit of Taiwan application serial no. 098139638, filed on Nov. 20, 2009. The entirety of the above-mentioned patent application is incorporated herein by reference and made a part of this specification.

### BACKGROUND

The present invention relates to a smart clothing for motion physiological measurement, and more particularly to a dynamical stable apparatus and a smart clothing for motion physiological measurement having the dynamical stable apparatus.

In clinical measurement, when applying an electrocardiogram apparatus to measure a physiological signal such as electrocardiogram, the electrocardiogram may be a static electrocardiogram or a dynamic electrocardiogram. When measuring the static electrocardiogram, a user who is measured lies on a bed. The measuring is carried out in a static state, the user is not allowed to move, and the user breathes slightly. When measuring the dynamic electrocardiogram, the user who is measured walks at a normal speed, and then speeds up to walk fast, jog, run at a middle speed and run at a fast speed. In a microelectrode technology for measuring the cardiac electrophysiological signal, a displacement of an electrode conductive clip caused by a movement of the user may result in noise or an abnormal signal. Moreover, due to a measuring portion of an electrode piece being coated with conductive adhesive, the user may feel uncomfortable or the user may be allergic, and therefore the electrocardiogram apparatus is not suitable for using for a long time.

Taiwanese Patent No. I274577 teaches a wearable physiological measuring system. The wearable physiological measuring system can be worn on a user so as to measure a physiological signal of the user. The wearable physiological measuring system has a tension adjusting device which can adjust a pressure force between a physiological sensor and the skin of the user according to different users, and thereby the physiological signal of the user can be correctly measured. A tension system of the wearable physiological measuring system can control a flexible binding force of the wearable physiological measuring system, however, when the wearable physiological measuring system is applied to a dynamic measurement, a noise or an abnormal signal is produced because of a slight displacement of a electrode conductive clip.

Therefore, what is needed is to provide a dynamical stable apparatus and a smart clothing for motion physiological measurement having the dynamical stable apparatus so as to obtain a stable motion physiological signal.

### BRIEF SUMMARY

The present invention provides dynamical stable apparatus applied to a smart clothing for motion physiological measurement so as to obtain a stable motion physiological signal of a user.

The present invention provides a smart clothing for motion physiological measurement so as to obtain a stable motion physiological signal of a user.

To achieve the above-mentioned advantages, the present invention provides a dynamical stable apparatus of a smart clothing for motion physiological measurement. The dynamical stable apparatus includes at least one physiological sensing module. Each physiological sensing module includes a carrier, at least one physiological sensor and a first tension adjusting component. The carrier has a surface for contacting with a body of a user, and the surface is coated with a slide resistant material. The at least one physiological sensor is disposed on the surface of the carrier and configured for measuring a motion physiological signal of the user. The first tension adjusting component is disposed on two opposite ends of the carrier and configured for adjusting a tension of the carrier so as to fix the carrier on the body of the user.

In one embodiment of the present invention, the carrier is a flexible belt textile and is suitable to surround the body of the user.

In one embodiment of the present invention, the surface includes a first area and a second area located on an area except the first area, the at least one physiological sensor is disposed on the first area, and the slide resistant material is coated on the first area.

In one embodiment of the present invention, the surface includes a first area and a second area located on an area except the first area, the at least one physiological sensor is disposed on the first area, a first slide resistant material is coated on the first area, a second slide resistant material is coated on the second area, and a friction force of the first area is larger or smaller than that of the second area.

In one embodiment of the present invention, the first slide resistant material and the second slide resistant material are the same, and a coating thickness of the first slide resistant material coated on the first area is larger or smaller than that of the second slide resistant material coated on the second area.

In one embodiment of the present invention, the first slide resistant material and the second slide resistant material are the same, and a coating density of the first slide resistant material coated on the first area is larger or smaller than that of the second slide resistant material coated on the second area.

In one embodiment of the present invention, the first slide resistant material and the second slide resistant material are different, and a friction force of the first slide resistant material coated on the first area is larger or smaller than that of the second slide resistant material coated on the second area.

In one embodiment of the present invention, the slide resistant material described is latex.

In one embodiment of the present invention, a coating thickness of the slide resistant material is ranged from 0.01 millimeters to 3 millimeters.

In one embodiment of the present invention, the coating thickness of the slide resistant material is ranged from 0.05 millimeters to 0.5 millimeters.

In one embodiment of the present invention, a coating manner for the slide resistant material is point-shaped, linear, surface-shaped or porous.

In one embodiment of the present invention, the slide resistant material has a plurality of micro-vents.

In one embodiment of the present invention, the first tension adjusting component is a Velcro belt or a buckling element.

In one embodiment of the present invention, the dynamical stable apparatus further includes at least one motion stable supporting member located on at least one side of an upper side and a lower side of the physiological sensing module, and the motion stable supporting member is suitable to surround the body of the user.

In one embodiment of the present invention, the motion stable supporting member includes a supporter and a second tension adjusting component. The second tension adjusting component is disposed on two opposite ends of the supporter, wherein the two opposite ends of the supporter are capable of being engaged with each other through the second tension adjusting component.

In one embodiment of the present invention, the supporter is a flexible textile which is rope-shaped, strip-shaped or ribbon-shaped, and the supporter is suitable to surround the body of the user.

In one embodiment of the present invention, the second tension adjusting component is a Velcro belt or a buckling element.

In one embodiment of the present invention, the supporter has a slide resistant surface for contacting with the body of the user, and the slide resistant surface is coated with a third slide resistant material.

To achieve the above-mentioned advantages, the present invention further provides a smart clothing for motion physiological measurement configured for measuring a motion physiological signal of a user. The smart clothing for motion physiological measurement includes a main body and a dynamical stable apparatus. The main body is configured to be worn on a body of the user and has an interior surface facing the body of the user. The dynamical stable apparatus is disposed on the interior surface of the main body and includes at least one physiological sensing module. Each physiological sensing module includes a carrier, at least one physiological sensor and a first tension adjusting component. The carrier has a surface for contacting with a body of the user, and the surface is coated with a slide resistant material. The physiological sensor is disposed on the surface of the carrier and configured for measuring the motion physiological signal of the user. The first tension adjusting component is disposed on two opposite ends of the carrier and configured for adjusting a tension of the carrier so as to fix the carrier on the body of the user.

In one embodiment of the present invention, the main body is a flexible textile and a longitude axis and a latitude axis of the flexible textile have flexibility.

To achieve the above-mentioned advantages, the present invention further provides a dynamical stable apparatus of a smart clothing for motion physiological measurement. The dynamical stable apparatus includes at least one physiological sensing module and at least one motion stable supporting member. Each physiological sensing module includes a carrier, at least one physiological sensor and a first tension adjusting component. The carrier has a surface for contacting with a body of a user. The physiological sensor is disposed on the surface of the carrier and configured for measuring a motion physiological signal of the user. The first tension adjusting component is disposed on two opposite ends of the carrier and configured for adjusting a tension of the carrier so as to fix the carrier on the body of the user. The motion stable supporting member is located on at least one side of an upper side and a lower side of the physiological sensing module and suitable to surround the body of the user.

In one embodiment of the present invention, each motion stable supporting member includes a supporter and a second tension adjusting component. The second tension adjusting component is disposed on two opposite ends of the supporter and the two opposite ends of the supporter are capable of being engaged with each other through the second tension adjusting component.

In one embodiment of the present invention, the supporter is rope-shaped, strip-shaped or ribbon-shaped and suitable to surround the body of the user.

In one embodiment of the present invention, the second tension adjusting component is a Velcro belt or a buckling element.

In one embodiment of the present invention, the supporter has a slide resistant surface for contacting with the body of the user, and the slide resistant surface is coated with a third slide resistant material.

In one embodiment of the present invention, the third slide resistant material is latex.

In one embodiment of the present invention, a coating thickness of the third slide resistant material is ranged from 0.01 millimeters to 3 millimeters.

In one embodiment of the present invention, the coating thickness of the third slide resistant material is ranged from 0.05 millimeters to 0.5 millimeters.

In one embodiment of the present invention, a coating manner for the third slide resistant material is point-shaped, linear, surface-shaped or porous.

In one embodiment of the present invention, the third slide resistant material has a plurality of micro-vents.

To achieve the above-mentioned advantages, the present invention further provides a smart clothing for motion physiological measurement configured for measuring a motion physiological signal of a user. The smart clothing for motion physiological measurement includes a main body and a dynamical stable apparatus. The main body is configured to be worn on a body of the user and has an interior surface facing the body of the user. The dynamical stable apparatus is disposed on the interior surface of the main body and includes at least one physiological sensing module and at least one motion stable supporting member. Each physiological sensing module includes a carrier, at least one physiological sensor and a first tension adjusting component. The carrier has a surface for contacting with the body of the user, and the surface is coated with a slide resistant material. The physiological sensor is disposed on the surface of the carrier and configured for measuring a motion physiological signal of the user. The first tension adjusting component is disposed on two opposite ends of the carrier and configured for adjusting a tension of the carrier so as to fix the carrier on the body of the user.

The smart clothing for motion physiological measurement of the present invention has the main body having the flexible binding force along a longitude axis and a latitude axis. When the user wearing the smart clothing for motion physiological measurement is in motion state, an involved movement of the dynamical stable apparatus caused by the smart clothing for motion physiological measurement and physiological noise or an abnormal signal caused by the involved movement can be reduced via the flexibility of the main body of the smart clothing for motion physiological measurement. The carrier of the dynamical stable apparatus has the surface for contacting with the body of the user. The surface is coated with the slide resistant material. The first tension adjusting component can adjust the tension of the carrier according to the stability and the comfort of the user when measuring the motion physiological signal. As such, when the user is in a motion state, a slippage between the physiological sensor of the dynamical stable apparatus and the skin of the user is prevented, thereby reducing a physiological noise or an abnormal signal caused by a movement of the dynamical stable apparatus. As a result, a stable motion physiological signal is obtained.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic view of a smart clothing for motion physiological measurement of a first embodiment of the present invention.

FIG. 2 is a schematic, exploded view of the smart clothing for motion physiological measurement of the first embodiment of the present invention.

FIG. 3 is a schematic view of a smart clothing for motion physiological measurement of a second embodiment of the present invention.

FIG. 4 is a schematic view of a smart clothing for motion physiological measurement of a third embodiment of the present invention.

FIG. 5 is a schematic view of a smart clothing for motion physiological measurement of a fourth embodiment of the present invention.

FIG. 6 is a schematic view of a smart clothing for motion physiological measurement of a fifth embodiment of the present invention.

### DETAILED DESCRIPTION

It is to be understood that other embodiment may be utilized and structural changes may be made without departing from the scope of the present invention. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings.

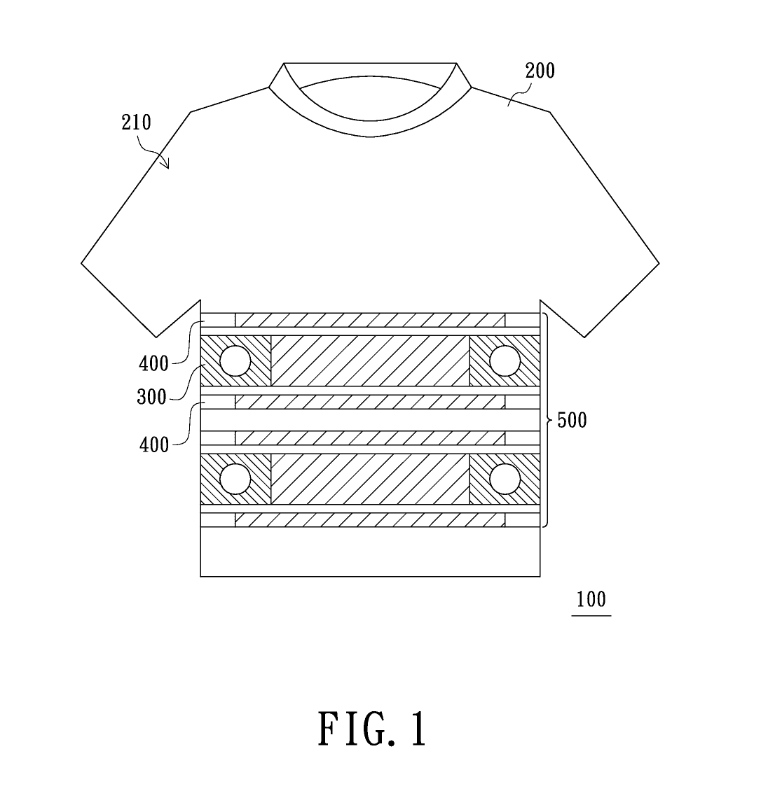

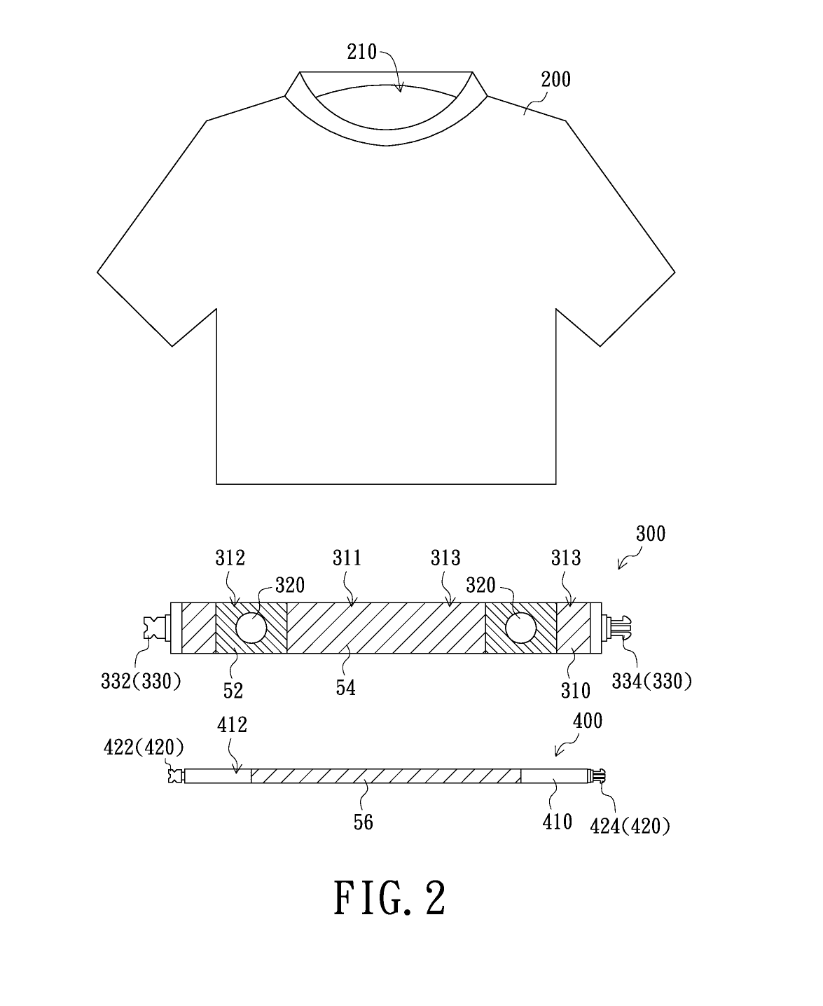

FIG. 1 is a schematic view of a smart clothing for motion physiological measurement of a first embodiment of the present invention. FIG. 2 is a schematic, exploded view of the smart clothing for motion physiological measurement of the first embodiment of the present invention. Referring to FIGS. 1 and 2, a smart clothing for motion physiological measurement 100 of the present embodiment includes a main body 200 and a dynamical stable apparatus 500.

The main body 200 is configured to be worn on a body of a user and has an interior surface 210 facing the body of the user. The main body 200 may be a flexible textile and a longitude axis and a latitude axis of the flexible textile have flexibility. More specifically, the main body 200 can be, but not limited to, a weave fabric, a knit fabric, or a nonwoven fabric, wherein the knit fabric is better. When the user wearing the smart clothing for motion physiological measurement 100 is in motion state, due to the main body 200 having flexible binding force along the longitude axis and the latitude axis, the main body 200 can reduce an involved movement of the dynamical stable apparatus 500 caused by the main body 200 and a physiological noise or an abnormal signal caused by the involved movement.

The dynamical stable apparatus 500 is disposed on the interior surface 210 of the main body 200. In order to clearly show all the components on the interior 210 of the smart clothing for motion physiological measurement 100, FIG. 1 shows the main body 200 inside out, that is, the interior surface 210 faces the viewer. The dynamical stable apparatus 500 is configured for measuring a motion physiological signal of the user, such as a cardiac electrophysiological signal and so on. The dynamical stable apparatus 500 includes at least one physiological sensing module 300 and at least one motion stable supporting member 400. The dynamical stable apparatus 500 of the present invention includes two physiological sensing modules 300 and four motion stable supporting members 400 corresponding to the physiological sensing modules 300. However, the present invention does not limit a number of the physiological sensing module 300 and the motion stable supporting member 400.

Each of the physiological sensing modules 300 includes a carrier 310, at least one physiological sensor 320 and a first tension adjusting component 330. In the present embodiment, two physiological sensors 320 are taken as an example, but a number of the physiological sensor 320 is not limited. In the embodiment, the carrier 310 may be, but not limited to, a flexible belt textile, and is suitable to surround the body of the user. The carrier 310 has a surface 311 for contacting with the body of the user and a back surface (not shown) opposite to the surface 311. The back surface is fixed to the interior surface 210 of the main body 200. The surface 311 includes a first area 312 and a second area 313 located on an area except the first area 312. In the present embodiment, the first area 312 of the surface 311 is composed of two independent areas which are not connected with each other and corresponds to a number of the physiological sensors 320. The second area 313 is located on the area except the first area 312. The second area 313 may be composed of two independent areas, and may also be an integrated area. In the present embodiment, the second area 313 is composed of two independent areas which separate the two independent areas of the first area 312. It should be pointed out that the present invention does not limit a shape and a position of the first area 312 and the second area 313.

The two physiological sensors 320 are respectively disposed on the two independent areas of the first area 312 of the surface 311 of the carrier 310. The physiological sensors 320 are configured for measuring the motion physiological signal of the user. The first area 312 of the surface 311 is coated with a first slide resistant material 52, and the second area 313 is coated with a second slide resistant material 54. A friction force of the first area 312 may be larger or smaller than that of the second area 313. In this way, the physiological sensors 320 can closely touch the skin of the user, which prevents a slippage between the physiological sensors 320 and the skin when the user is in motion state, thereby reducing a physiological noise or an abnormal signal caused by a displacement of the physiological sensors 320 due to a movement of the user. Therefore, the smart clothing for motion physiological measurement 100 can obtain a stable motion physiological signal of the user.

In particular, when the first slide resistant material 52 and the second slide resistant material 54 are the same, a coating density of the first slide resistant material 52 coated on the first area 312 can be larger than that of the second slide resistant material 54 coated on the second area 313, or a coating thickness of the first slide resistant material 52 coated on the first area 312 can be larger than that of the second slide resistant material 54 coated on the second area 313. The above two examples can achieve that the friction force of the first area 312 is larger than that of the second area 313. In addition, when the first slide resistant material 52 and the second slide resistant material 54 are different, the first slide resistant material 52 and the second slide resistant material 54 can have different friction forces. That is, the friction force of the first slide resistant material 52 coated on the first area 312 is larger than that of the second slide resistant material 54 coated on the second area 313.

On the contrary, when the first slide resistant material 52 and the second slide resistant material 54 are the same, the coating density of the first slide resistant material 52 coated on the first area 312 can be smaller than that of the second slide resistant material 54 coated on the second area 313, or the coating thickness of the first slide resistant material 52 coated on the first area 312 can be smaller than that of the second slide resistant material 54 coated on the second area 313. The above two examples can achieve that the friction force of the first area 312 is smaller than that of the second area 313. In addition, when the first slide resistant material 52 and the second slide resistant material 54 are different, the first slide resistant material 52 and the second slide resistant material 54 can have different friction forces. That is, the friction force of the first slide resistant material 52 coated on the first area 312 is smaller than that of the second slide resistant material 54 coated on the second area 313.

The first slide resistant material 52 and the second slide resistant material 54 may be, but not limited to, latex. Additionally, the first slide resistant material 52 and the second slide resistant material 54 may have a plurality of micro-vents which can improve the permeability of the carrier 310, thereby improving comfort for the user. The coating thickness of the first slide resistant material 52 and the second slide resistant material 54 may be ranged from 0.01 millimeters to 3 millimeters and preferably ranged from 0.05 millimeters to 0.5 millimeters.

A coating manner for the first slide resistant material 52 and the second slide resistant material 54 may be, but not limited to, point-shaped, linear, surface-shaped or porous. FIG. 2 of the present embodiment shows that the first slide resistant material 52 and the second slide resistant material 54 are coated by the surface-shaped manner, and each of the first slide resistant material 52 and the second slide resistant material 54 has the micro-vents. The coating density of the first slide resistant material 52 coated on the first area 312 is larger than that of the second slide resistant material 54 coated on the second area 313, so that the friction force of the first area 312 is larger than that of the second area 313.

The first tension adjusting component 330 is disposed on two opposite ends of the carrier 310 and configured for adjusting a tension of the carrier 310 so as to fix the carrier 310 on the body of the user. In the present embodiment, the first tension adjusting component 330 can be a Velcro belt or a buckling element. Two corresponding components 332 and 334 which constitute the Velcro belt or the buckling element are disposed on two opposite ends of the carrier 310. In addition, in the present embodiment, the first tension adjusting component 330 is the buckling element, the corresponding components 332 and 334 are respectively a male buckling element and a female buckling element which can engage with each other. However, the first tension adjusting component 330 is not limited by this example, as long as the first tension adjusting component 330 can adjust the tension of the carrier 310 according to the stability and the comfort of the user when measuring the motion physiological signal, so as to prevent the slippage between the physiological sensors 320 of the carrier 310 and the skin when the user is in motion state, thereby reducing the physiological noise or the abnormal signal.

The motion stable supporting member 400 is located on at least one side of an upper side and a lower side of the physiological sensing module 300. In the present embodiment, four motion stable supporting members 400 are taken as an example. The four motion stable supporting members 400 are disposed on an interior surface 210 of the main body 200, and every two motion stable supporting members 400 are respectively located on the upper side and the lower side of the physiological sensing module 300.

The motion stable supporting members 400 can be in contact with the physiological sensing module 300 and can be spaced from the physiological sensing module 300. The motion stable supporting members 400 are suitable to surround the body of the user. When the user puts on the smart clothing for motion physiological measurement 100 and is in motion state, the motion stable supporting members 400 can further reduce the involved movement of the physiological sensing module 300 caused by the main body 200 of the smart clothing for motion physiological measurement 100, and also reduce the physiological noise or the abnormal signal caused by the involved movement.

In the present embodiment, each motion stable supporting member 400 includes a supporter 410 and a second tension adjusting component 420. The supporter 410 is a flexible textile which may be, but not limited to, rope-shaped, strip-shaped or ribbon-shaped. The supporter 410 is suitable to surround the body of the user and has a slide resistant surface 412 for contacting with the body of the user. In the present embodiment, the slide resistant surface 412 may be coated with a third slide resistant material 56. The third slide resistant material 56 can be, but not limited to, latex. Furthermore, the third slide resistant material 56 may have a plurality of micro-vents so as to improve the permeability of the supporter 410 and comfort for the user. A coating thickness of the third slide resistant material 56 may be ranged from 0.01 millimeters to 3 millimeters and preferably ranged from 0.05 millimeters to 0.5 millimeters. A coating manner for the third slide resistant material 56 may be point-shaped, linear, surface-shaped or porous. FIG. 2 of the present embodiment shows that the third slide resistant material 56 is coated by the surface-shaped manner, and each of the third slide resistant material 56 has the micro-vents.

The second tension adjusting component 420 is configured for adjusting a tension of the supporter 410 so as to fix the supporter 410 on the body of the user. In the present embodiment, the second tension adjusting component 420 can be a Velcro belt or a buckling element. Two corresponding components 422 and 424 which constitute the Velcro belt or the buckling element are disposed on two opposite ends of the supporter 410. The second tension adjusting component 420 is not limited to the Velcro belt or the buckling element, as long as the supporter 410 can adjust the tension and the two opposite ends of the supporter 410 can engage with each other. Furthermore, the second tension adjusting component 420 of the motion stable supporting member 400 can be omitted because the two opposite ends of the supporter 410 can be directly tied. Additionally, the motion stable supporting member 400 can also be disposed on an outer surface of the main body 200.

It should be pointed out that the present invention does not limit that the surface 311 of the physiological sensing modules 300 should be coated with slide resistant material. In such case, the involved movement of the physiological sensing module 300 caused by the main body 200 of the smart clothing for motion physiological measurement 100 and the physiological noise or the abnormal signal caused by the involved movement can be reduced by the motion stable supporting members 400. The surface 311 of the carrier 310 can be selectively coated with the slide resistant material so as to reduce the physiological noise or the abnormal signal caused by the displacement of the dynamical stable apparatus 500 of the physiological sensing module 300 due to the movement of the user.

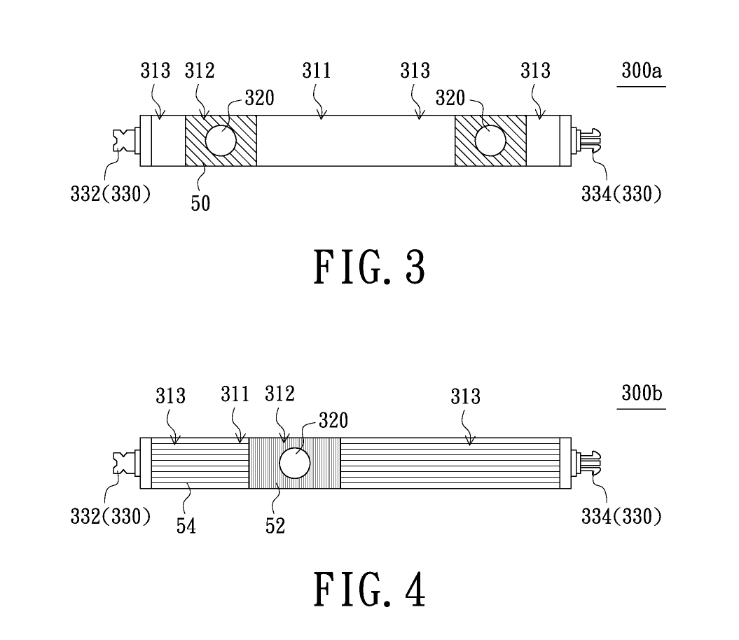

FIG. 3 is a schematic view of a smart clothing for motion physiological measurement of a second embodiment of the present invention. Referring to FIG. 3, a physiological sensing module 300a of the present embodiment is similar to the physiological sensing module 300 of the first embodiment, wherein the difference is that the slide resistant material 50 is only coated on the first area 312 and is not coated on the second area 313. In the present embodiment, a friction force of the first area 312 is larger than that of the second area 313. Furthermore, the slide resistant material 50 may be, but not limited to, latex. The slide resistant material 50 may include micro-vents which can improve the permeability of the first area 312, thereby increasing comfort for the user. The coating thickness of the slide resistant material 50 may be ranged from 0.01 millimeters to 3 millimeters and preferably ranged from 0.05 millimeters to 0.5 millimeters. In addition, the coating manner for the slide resistant material 50 may be, but not limited to, point-shaped, linear, surface-shaped or porous.

FIG. 4 is a schematic view of a smart clothing for motion physiological measurement of a third embodiment of the present invention. Referring to FIG. 4, a physiological sensing module 300b of the present embodiment is similar to the physiological sensing module 300 of the first embodiment, wherein the difference is that a physiological sensing module 300b only includes a single physiological sensor 320. In the present embodiment, the surface 311 includes the first area 312 and the second area 313. The first area 312 is configured for arranging the physiological sensor 320, and the second area 313 is located on an area except the first area 312. In addition, the first slide resistant material 52 coated on the first area 312 and the second slide resistant material 54 coated on the second area 313 are formed by linear coating manner, and each of the first slide resistant material 52 and the second slide resistant material 54 has the micro-vents. The coating thickness of the first slide resistant material 52 may be larger or smaller than that of the second slide resistant material 54 such that the friction force of the first area 312 is larger or smaller than the friction force of the second area 313. Additionally, similarly to the first embodiment, the first slide resistant material 52 and the second resistant material 54 may be the same or different.

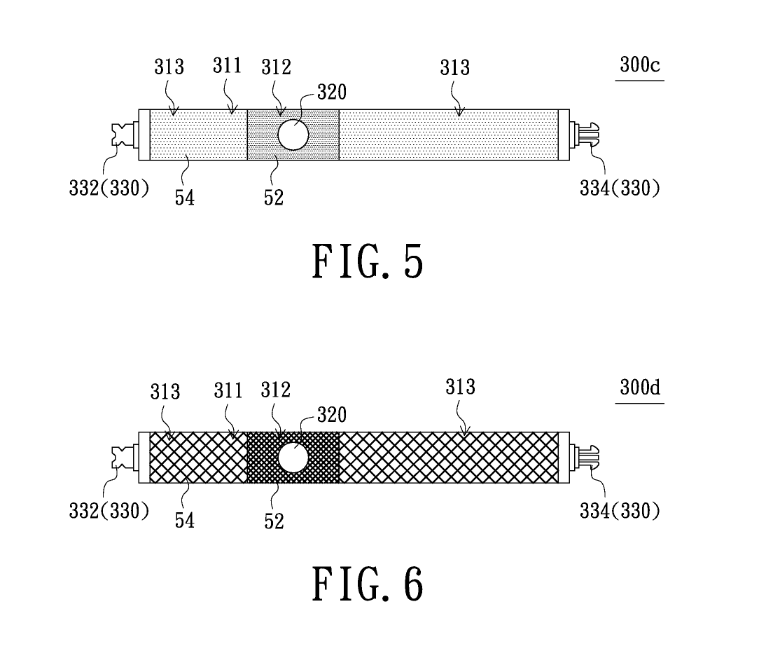

FIG. 5 is a schematic view of a smart clothing for motion physiological measurement of a fourth embodiment of the present invention. Referring to FIG. 5, a physiological sensing module 300c of the present embodiment is similar to the physiological sensing module 300b of the third embodiment, wherein the difference is that the first slide resistant material 52 coated on the first area 312 and the second slide resistant material 54 coated on the second area 313 are formed by point-shaped coating manner and each of the first slide resistant material 52 and the second slide resistant material 54 has the micro-vents. The coating density of the first slide resistant material 52 may be larger or smaller than that of the second slide resistant material 54 such that the friction force of the first area 312 is larger or smaller than that of the second area 313. Additionally, similarly to the first embodiment, the first slide resistant material 52 and the second resistant material 54 may be the same or different.

FIG. 6 is a schematic view of a smart clothing for motion physiological measurement of a fifth embodiment of the present invention. Referring to FIG. 6, a physiological sensing module 300d of the present embodiment is similar to the physiological sensing module 300b of the third embodiment, wherein the difference is that the first slide resistant material 52 coated on the first area 312 and the second slide resistant material 54 coated on the second area 313 are formed by porous coating manner, and the coating density of the first slide resistant material 52 may be larger or smaller than that of the second slide resistant material 54 such that the friction force of the first area 312 is larger or smaller than that of the second area 313. Additionally, similarly to the first embodiment, the first slide resistant material 52 and the second resistant material 54 may be the same or different.

It should be pointed out that, in every embodiment described above, the coating manner of the first slide resistant material 52 and the coating manner of the second slide resistant material 54 are the same, however, in other embodiments, the coating manner of the first slide resistant material 52 and the coating manner of the second slide resistant material 54 may be different.

In summary, when the user puts on the smart clothing for motion physiological measurement to measure the motion physiological signals, since the surface of the carrier is coated with the slide resistant material and the physiological sensing module further cooperates with the motion stable supporting members, the displacement of the dynamical stable apparatus is effectively prevented, thereby promoting the stability of the motion physiological signal. As a result, the stable motion physiological signal is obtained. Furthermore, the smart clothing for motion physiological measurement and the dynamical stable apparatus thereof of the present invention have the slide resistant material and the slide resistant material has the micro-vents, which cooperates with the usage of the tension adjusting components so that the comfort for the user is improved.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A dynamical stable apparatus of a smart clothing for motion physiological measurement, comprising:
at least one physiological sensing module, each physiological sensing module comprising:
a carrier having a surface for contacting with a body of a user, the surface being coated with a slide resistant material;
at least one physiological sensor disposed on the surface of the carrier and configured for measuring a motion physiological signal of the user; and
a first tension adjusting component disposed on two opposite ends of the carrier and configured for adjusting a tension of the carrier to fix the carrier on the body of the user.

2. The dynamical stable apparatus as claimed in claim 1, wherein the carrier is a flexible belt textile and is suitable to surround the body of the user, the slide resistant material is latex, a coating thickness of the slide resistant material is ranged from 0.01 millimeters to 3 millimeters, a coating manner for the slide resistant material is point-shaped, linear, surface-shaped or porous, the slide resistant material has a plurality of micro-vents, and the first tension adjusting component is a Velcro belt or a buckling element.

3. The dynamical stable apparatus as claimed in claim 1, wherein the surface includes a first area and a second area located on an area except the first area, the at least one physiological sensor is disposed on the first area, and the slide resistant material is coated on the first area.

4. The dynamical stable apparatus as claimed in claim 1, wherein the surface includes a first area and a second area located on an area except the first area, the at least one physiological sensor is disposed on the first area, the slide resistant material comprises a first slide resistant material coated on the first area and a second slide resistant material coated on the second area, and a friction force of the first area is larger or smaller than that of the second area.

5. The dynamical stable apparatus as claimed in claim 4, wherein the first slide resistant material and the second slide resistant material are the same, and a coating thickness of the first slide resistant material coated on the first area is larger or smaller than that of the second slide resistant material coated on the second area.

6. The dynamical stable apparatus as claimed in claim 4, wherein the first slide resistant material and the second slide resistant material are the same, and a coating density of the first slide resistant material coated on the first area is larger or smaller than that of the second slide resistant material coated on the second area.

7. The dynamical stable apparatus as claimed in claim 4, wherein the first slide resistant material and the second slide resistant material are different, and a friction force of the first slide resistant material coated on the first area is larger or smaller than that of the second slide resistant material coated on the second area.

8. The dynamical stable apparatus as claimed in claim 1, further comprising at least one motion stable supporting member located on at least one side of an upper side and a lower side of the physiological sensing module, and the motion stable supporting member being suitable to surround the body of the user.

9. The dynamical stable apparatus as claimed in claim 8, wherein each motion stable supporting member comprising:
a supporter; and
a second tension adjusting component disposed on two opposite ends of the supporter, wherein the two opposite ends of the supporter are capable of being engaged with each other through the second tension adjusting component.

10. The dynamical stable apparatus as claimed in claim 9, wherein the supporter is a textile which is rope-shaped, strip-shaped or ribbon-shaped, the supporter is suitable to surround the body of the user, the second tension adjusting component is a Velcro belt or a buckling element, the supporter has a slide resistant surface for contacting with the body of the user, the slide resistant surface is coated with a third slide resistant material, the third slide resistant material is latex, a coating thickness of the third slide resistant material is ranged from 0.01 millimeters to 3 millimeters, a coating manner for the third slide resistant material is point-shaped, linear, surface-shaped or porous, and the third slide resistant material has a plurality of micro-vents.

11. A smart clothing for motion physiological measurement configured for measuring a motion physiological signal of a user, comprising:
a main body configured to be worn on a body of the user and having an interior surface facing the body of the user; and
a dynamical stable apparatus of a smart clothing for motion physiological measurement claimed in claim 1, disposed on the interior surface of the main body.

12. The smart clothing for motion physiological measurement as claimed in claim 11, wherein the main body is a flexible textile and a longitude axis and a latitude axis of the flexible textile have flexibility.

13. A dynamical stable apparatus of a smart clothing for motion physiological measurement, comprising:
at least one physiological sensing module, each physiological sensing module comprising:
a carrier having a surface for contacting with a body of a user;
at least one physiological sensor disposed on the surface of the carrier and configured for measuring a motion physiological signal of the user; and
a first tension adjusting component disposed on two opposite ends of the carrier and configured for adjusting a tension of the carrier to fix the carrier on the body of the user, and
at least one motion stable supporting member located on at least one side of an upper side and a lower side of the physiological sensing module, and the at least one motion stable supporting member being suitable to surround the body of the user.

14. The dynamical stable apparatus as claimed in claim 13, wherein the first tension adjusting component is a Velcro belt or a buckling element, each motion stable supporting member includes a supporter and a second tension adjusting component, the second tension adjusting component is disposed on two opposite ends of the supporter and the two opposite ends of the supporter are capable of being engaged with each other through the second tension adjusting component, the supporter is rope-shaped, strip-shaped or ribbon-shaped and suitable to surround the body of the user, the second tension adjusting component is a Velcro belt or a buckling element, the supporter has a slide resistant surface for contacting with the body of the user, the slide resistant surface is coated with a third slide resistant material, a coating thickness of the third slide resistant material is ranged from 0.01 millimeters to 3 millimeters, a coating manner for the third slide resistant material is point-shaped, linear, surface-shaped or porous, and the third slide resistant material has a plurality of micro-vents.

15. A smart clothing for motion physiological measurement configured for measuring a motion physiological signal of a user, comprising:
a main body configured to be worn on a body of the user and having an interior surface facing the body of the user; and
a dynamical stable apparatus of a smart clothing for motion physiological measurement disposed on the interior surface of the main body and comprising:
at least one physiological sensing module, each physiological sensing module comprising:
a carrier having a surface for contacting with a body of a user;
at least one physiological sensor disposed on the surface of the carrier and configured for measuring a motion physiological signal of the user; and
a first tension adjusting component disposed on two opposite ends of the carrier and configured for adjusting a tension of the carrier to fix the carrier on the body of the user, and
at least one motion stable supporting member located on at least one side of an upper side and a lower side of the physiological sensing module, and the at least one motion stable supporting member being suitable to surround the body of the user.

16. The smart clothing for motion physiological measurement as claimed in claim 15, wherein the main body is a flexible textile and a longitude axis and a latitude axis of the flexible textile have flexibility.
